(19) United States
(12) Patent Application Publication
(10) Pub. No.: US 2017/0073306 A1
(43) Pub. Date: Mar. 16, 2017

(54) POTENT INHIBITORS OF HUMAN CARBONIC ANHYDRASE II AND BOVINE CARBONIC ANHYDRASE II AND THEIR MECHANISM OF ACTION

(71) Applicants: Muhammad Iqbal Choudhary, Karachi (PK); Muhammad Saleem, Karachi (PK); Aamer Saeed Bhatti, Islamabad (PK); Atia-tul Wahab, Karachi (PK); Ajmal Khan, Abbottabad (PK); Atta-ur- Rahman, Karachi (PK)

(72) Inventors: Muhammad Iqbal Choudhary, Karachi (PK); Muhammad Saleem, Karachi (PK); Aamer Saeed Bhatti, Islamabad (PK); Atia-tul Wahab, Karachi (PK); Ajmal Khan, Abbottabad (PK); Atta-ur- Rahman, Karachi (PK)

(21) Appl. No.: 14/854,981

(22) Filed: Sep. 15, 2015

(51) Int. Cl. C07C 335/26; C07D 307/24

(52) U.S. Cl. CPC C07C 335/26; C07D 307/24

(57) ABSTRACT

Inhibitors of carbonic anhydrase-II derived from sulfanilamide are reported.

### BACKGROUND OF THE INVENTION

Mechanism based high throughput calorimetric method is a significant procedure for the characterization of the enzymes reaction, and origin of their catalysis in bio-industry. Enzyme inhibition defines, “the interactions of enzyme-substrate or enzyme-inhibitor or both in the moiety of active site”. In recent years, application of enzymes their inhibition have made tremendous progress in the field of healthcare, pharmaceutical, bio-industries, environment, and biochemical enzyme chip industries. Calorimetric enzyme technology has given a new way of drug discovery. Every year new enzyme inhibitors are discovered as useful drugs but success still needs to minimize challenges. However, detoxification or reduced toxic effects of many antitoxins are also accomplished mainly due to their enzyme inhibitory action. Therefore, studying the aforesaid enzyme kinetics and structure-activity relationship is vital to understand the kinetics of enzyme inhibition that in turn is fundamental to the modern design of pharmaceuticals industries (Sami and Shakoor, 2011).

The super family of zinc metallo-enzymes carbonic anhydrases (CAs, EC 4.2.1.1) are reported from a variety of organisms (Supuran, C T and Scozzafava, A, 2001, 2008). These Zn-metallo-enzymes catalyze the inter conversion of carbon dioxide and bicarbonate, an essential physiological reaction (Temperini, C et. al, 2008, Smith K S and Ferry J G, 2000, Masereel B et. al 2006). Many physiological processes, such as transportation of carbon dioxide from lungs to the metabolizing tissue, homeostasis in pH and other pathological or physiological reactions are catalyzed by the carbonic anhydrases ( [symbol] apkauskaite E, et. al 2012, Nishimori I, et. al 2007, Vullo D, et. al 2004).

In mammals, there are 16 different CA iso-zymes or CA related proteins (CARP): several cytosolic forms (such as CA I-III and CA-VII), five membrane-bound iso-zymes (CA-IV, CA-IX, CA-XII, CA-XIV and CA-XV), one mitochondrial form (CA-V), as well as a secreted CA iso-zymes (CA-VI) (Vullo D, et. al 2005). Recently, two tumor-associated membrane carbonic anhydrase iso-zymes (CA-IX and CA-XII) attract much more attention. The role of CA-IX in tumor physiology has been reported, such as the control of tumor pH and the influence in the cell microenvironment that promote cell proliferation, invasion, and metastasis (Gilmour K M, 2010, Vullo D et. al, 2003, Scozzafava A et. al, 2005). For this purpose major efforts have been made to design anticancer drugs against CA-IX. Although there are many effective inhibitors of CAs, most of classical sulfonamides/sulfamates CA inhibitors and their derivatives, such as acetazolamide (AZA), ethoxzolamide (EZA), do not have a good selectivity of CA-IX, over the sulfonamide-acid isozyme CA-II. Therefore, the challenge in the development of CA-II inhibitors is to identify highly selective inhibitors. In this study, nineteen novel scaffolds from the commercial SPECS database were identified with potent inhibitory activities against BCA-II and HCA-II with IC₅₀ values ranging between 0.060±0.002 to 0.28±0.03 μM and 0.1±0.04 to 0.58±0.001 μM by employing calorimetric mechanism based bioassay screening. The chemistry of these compounds is published (Saeed A et al, 2013). This is latest biological activities on this class of compound with special emphasis on the carbonic anhydrase-II inhibitory activity.

### BRIEF SUMMARY OF THE INVENTION

Inhibition of CAs iso-zymes by aromatic/heterocyclic sulfonamides has been used clinically for the treatment of a variety of diseases, such as glaucoma, epilepsy, congestive heart failure, mountain sickness, gastric and, duodenal ulcers, etc. Sulfonamide CA inhibitor was the first non-comer curial diuretic in clinical use in the early 1950s which subsequently led to the development of the thiazide and high-ceiling diuretics, two classes of widely used pharmacological agents. Due to a wide range of pharmacological activates of sulfonamide, their role as carbonic anhydrase inhibitors have been widely studied. Many drugs are available on the market, but there is a lack of specificity among different iso-zymes as well as different source enzymes.

Our invention can lead to the discovery of some specific inhibitors of HCA-II and BCA-II.

### BRIEF DESCRIPTION OF THE DRAWINGS

FIG. 1 depicts the structures of benzamide sulfonamide.

FIG. 2A depicts the Lineweaver-Burk plot of compound 2, in which 1/s (inverse of substrate concentrations) is plotted on x-axis, while 1/v (inverse of Vₘₐₓ values) is plotted on y-axis. Figure shows that apparent Km of the enzyme remains unaffected while Vₘₐₓ has been decreased i.e., it is noncompetitive inhibitor.

FIG. 2B depicts the double reciprocal plot for Ki determination which is between Slope Vs Inhibitor Conc.

FIG. 2C depicts the Dixon plot which is plotted between 1/Vₘₐₓ and [I] which confirms the Ki and type of inhibition which is noncompetitive for compound 2 against BCA-II.

FIG. 3A depicts the lineweaver-Burk plot of compound 19, in which 1/s (inverse of substrate concentrations) is plotted on x-axis, while 1/v (inverse of Vₘₐₓ values) is plotted on y-axis. Figure shows that apparent Km and Vₘₐₓ have been change i.e it is mixed-type inhibitor.

FIG. 3B depicts the double reciprocal plot for Ki determination which is between Slope Vs Inhibitor Conc.

FIG. 3C depicts the Dixon plot there which plotted between 1/Vₘₐₓ and [I], which indicate type of inhibition as non-competitive against BCA-II.

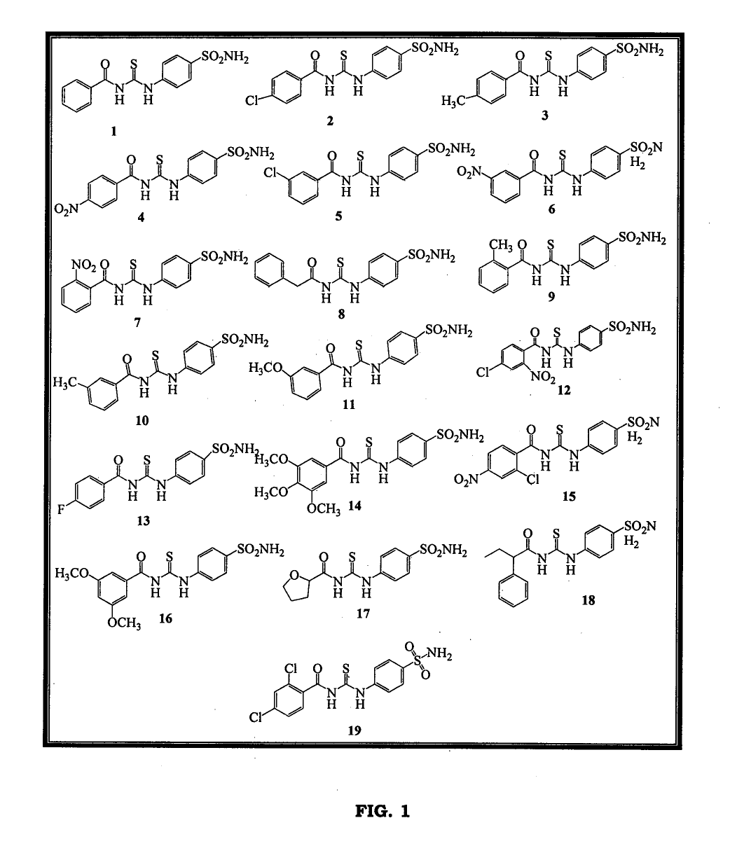

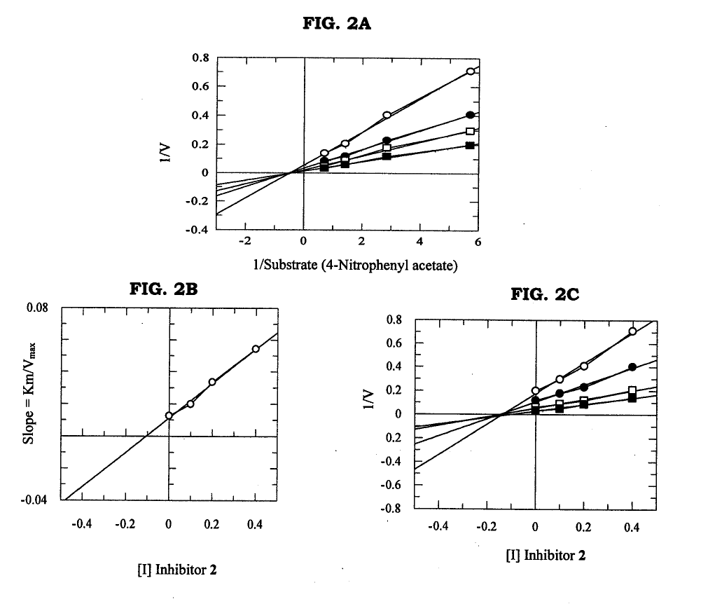

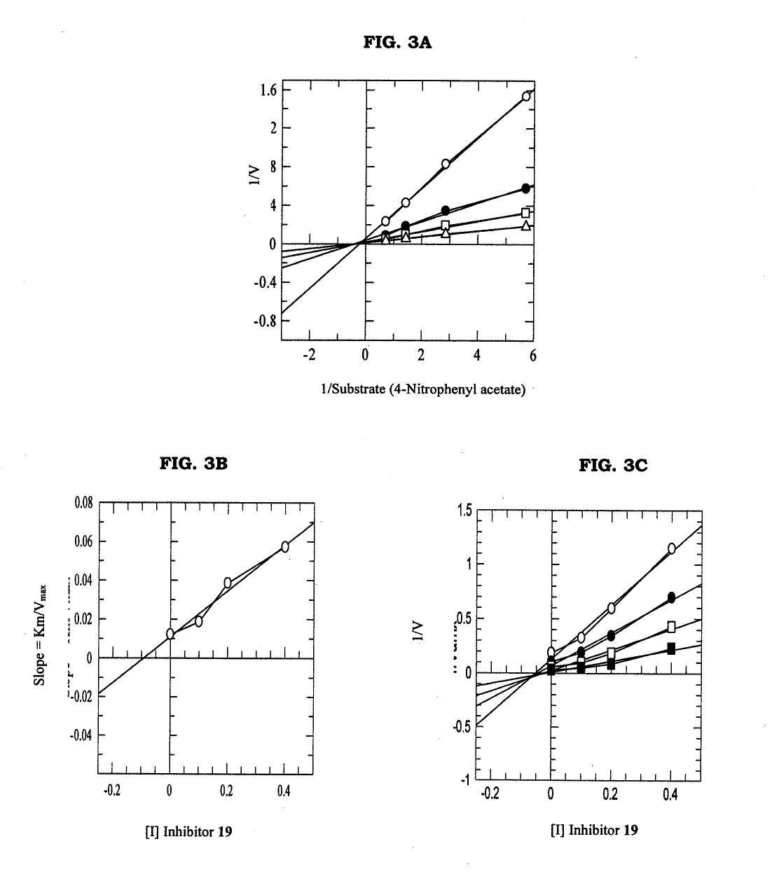

### DETAILED DESCRIPTION OF THE INVENTION

The discovery of these compounds as potent inhibitors of BCA-II and HCA-II can lead to a therapeutic intervention to treat carbonic anhydrase-II associated disorders such as glaucoma, leukemia, epilepsy and cystic fibrosis. The procedure employed to discover these inhibitors was simple and reliable. We have used spectrophotometric assay protocol for these discoveries.

### In-Vitro Inhibition of Carbonic Anhydrase-II

Nineteen different analogues of benzamide sulfonamides were evaluated for carbonic anhydrase-II inhibition. The rate of catalyzed reaction was monitored by the amount of product formation by using the chromogenic substrate i.e., 4-nitrophenyl acetate. All the analogues posses comparable inhibition potential when compared with the standard drugs such as acetazolamide and zonisamide (IC₅₀=0.12±0.03 and 1.86±0.03 μM, respectively). Among these derivatives compounds 1 and 19 were identified as potent inhibitors of both isozymes (BCA-II, HCA-II) with IC₅₀ values of 0.09±0.007, 0.11±0.02 and 0.06±0.001, 0.1±0.04 μM respectively. The inhibition potential of these analogues is presented in Table-1.

TABLE 1
Inhibition potential of benzamide sulfonamide derivatives against BCA-II and HCA-II

| | | IC₅₀ ± S.E.M. [μM] | |
|---|---|---|---|
| No. | IUPAC NAMES | BCA-II | HCA-II |
| 1 | N-(4-Sulfamoylphenylcarbamothioyl)benzamide | 0.09 ± 0.007 | 0.11 ± 0.02 |
| 2 | 4-Chloro-N-(4-sulfamoylphenylcarbamothioyl)benzamide | 0.16 ± 0.002 | 0.21 ± 0.01 |
| 3 | 4-Methyl-N-(4-sulfamoylphenylcarbamothioyl)benzamide | 0.08 ± 0.003 | 0.09 ± 0.03 |
| 4 | 4-Nitro-N-(4-sulfamoylphenylcarbamothioyl)benzamide | 0.13 ± 0.002 | 0.24 ± 0.04 |
| 5 | 3-Chloro-N-(4-sulfamoylphenylcarbamothioyl)benzamide | 0.14 ± 0.006 | 0.28 ± 0.06 |
| 6 | 3-Nitro-N-(4-sulfamoylphenylcarbamothioyl)benzamide | 0.08 ± 0.004 | 0.19 ± 0.001 |
| 7 | 2-Nitro-N-(4-sulfamoylphenylcarbamothioyl)benzamide | 0.06 ± 0.005 | 0.12 ± 0.002 |
| 8 | 2-Phenyl-N-(4-sulfamoylphenylcarbamothioyl)acetamide | 0.07 ± 0.003 | 0.16 ± 0.001 |
| 9 | 2-Methyl-N-(4-sulfamoylphenylcarbamothioyl)benzamide | 0.08 ± 0.002 | 0.18 ± 0.004 |
| 10 | 3-Methyl-N-(4-sulfamoylphenylcarbamothioyl)benzamide | 0.09 ± 0.002 | 0.19 ± 0.00 |
| 11 | 3-Methoxy-N-(4-sulfamoylphenylcarbamothioyl)benzamide | 0.09 ± 0.003 | 0.19 ± 0.005 |
| 12 | 4-Chloro-2-nitro-N-(4-sulfamoylphenylcarbamothioyl)benzamide | 0.28 ± 0.007 | 0.46 ± 0.03 |
| 13 | 4-Fluoro-N-(4-sulfamoylphenylcarbamothioyl)benzamide | 0.13 ± 0.02 | 0.58 ± 0.001 |
| 14 | 3,4,5-Trimethoxy-N-(4- | 0.06 ± 0.00 | 0.17 ± 0.002 |
| | sulfamoylphenylcarbamothioyl)benzamide | | |
| 15 | 2-Chloro-4-nitro-N-(4-sulfamoylphenylcarbamothioyl)benzamide | 0.06 ± 0.001 | 0.16 ± 0.01 |
| 16 | 3,5-Dimethoxy-N-(4-sulfamoylphenyl carbamothioyl)benzamide | 0.11 ± 0.005 | 0.23 ± 0.03 |
| 17 | N-(4-Sulfamoylphenylcarbamothioyl)tetrahydrofuran-2- | 0.12 ± 0.02 | 0.27 ± 0.06 |
| | carboxamide | | |
| 18 | 2-Phenyl-N-(4-sulfamoylphenylcarbamothioyl)butanamide | 0.2 ± 0.001 | 0.25 ± 0.01 |
| 19 | 2,4-Dichloro-N-(4-sulfamoylphenylcarbamothioyl)benzamide | 0.06 ± 0.001 | 0.1 ± 0.04 |
| ACZ | N-(5-Sulfamoyl-1,3,4-thiadiazol-2-yl)-acetamide | 0.12 ± 0.03 | 0.09 ± 0.02 |

S.E.M. = Standard Error of Mean,
ACZ = acetazolamide (standard inhibitor),
BCA-II = Bovine Carbonic Anhydrase-II,
HCA-II = Human Carbonic Anhydrase-II,
IC₅₀ = Minimum inhibitory Concentration.

SAR revealed that activity of the compounds is mainly due to sulfonamide functionality. The activity may be increased or decreased by different substituents on the benzamide aromatic system. The parent compound 1 have shown IC₅₀ value of 0.09±0.007 μM against BCA-II and 0.11±0.02 μM against HCA-II. In case of compound 19 it is dichloro substituted on the benzamide and exhibit IC₅₀ value of 0.06±0.001 μM against BCA-II and 0.1±0.04 μM against HCA-II. The strong electron donating effect of Cl— group has enhanced the inhibition potential against the both source enzymes.

### Assay Protocols of Bovine Carbonic Anhydrase Inhibition

4-Nitrophenyl acetate (4-NPA) is a colorless compound which is used as a substrate in carbonic anhydrase inhibition assay. The reaction involves the hydrolysis of 4-nitrophenyl acetate into CO₂ and 4-nitrophenol a yellow colored compound. During this experiment, HEPES-tris buffer was used at 20 mM concentration with pH 7.4. Total reaction volume of each well was 200 μL, which contain 140 μL HEPES-tris buffer, 20 μL of freshly prepared pure bovine erythrocyte CA-II (prepared in de-ionized water 0.1 mg/mL for 96-well flat bottom plate), 20 μL of test compounds prepared in DMSO at total concentration of 10%, 20 μL of chromogenic substrate (4-nitrophenyl acetate) at a concentration of 0.7 mM diluted in 95% ethanol.

After 15 min of incubation of the enzyme with the test compound, the substrate was added to initiate the reaction. The reaction was carried out in triplicate with different concentrations of test compounds was used.

In this assay protocol, the reaction was performed by using a 96-well flat bottom plate.

The amount of product formation was measured at 400 nm for 30 min with 1 min interval, multiplate reader SPECTRA max 340 (Molecular Devices, CA, USA) was used to monitor the product formation.

### 2.3 Assay Procedure for HCA-II Inhibitory Activity

Hydrolysis of 4-NPA (a colorless compound): yields 4-nitrophenol and CO₂. The reaction is monitored by quantifying the formation of 4-nitrophenol, a yellow colored compound.

In successive experiments, HEPES-tris was used as a buffer for the reaction at final concentration of 20 mM, with 7.4 pH. The total reaction volume of 200 μL includes, 140 μL of the HEPES-tris, 20 μL of test compound diluted in DMSO (20 μL of DMSO as a control sample), 20 μL of purified human erythrocyte CA-II (0.1 mg/mL) prepared in deionized water and 20 μL of a solution of 4-NPA (0.7 mM diluted in ethanol). Adhesion of enzyme with the plastic-ware was avoided by the addition of bovine serum albumin (1 mg/mL) [100].

The 20 μL of test compound was incubated with enzyme for a period of 15 min. The rate of product formation was monitored with the addition of 20 μL substrate prepared in ethanol at a final concentration of 0.7 mM 4-nitrophenyl acetate at 25° C. for a period of 30 min with regular interval of 1 min. M2 and Spectra Max 384 (Molecular Devices, CA, USA) were used for the measurement of product formation at a wavelength of 400 nm.

The assay was performed in triplicates by using 96-well flat bottom plate.

### Cytotoxicity Studies

Cytotoxicity activity of active compounds was evaluated by using the standard MTT (3-[4,5-dimethylthiazole-2-yl]-2,5-diphenyl-tetrazolium bromide) colorimetric assay in 96-well plate²³.

### Principle

Cytotoxicity studies were performed using a colorimetric assay that measures the reduction of 3-(4,5-dimethythiazol-2-yl)-2,5-diphenyl tetrazolium bromide (MTT) (yellow in color), by mitochondrial enzyme (i.e. succinate dehydrogenase). The MTT enters into the mitochondria of cell, where it is reduced to an insoluble formazan salt (purple in color). The extent of MTT reduction to formazan within cells was measured spectrophotometrically. As reduction of MTT can only occur in metabolically active cells the level of activity is actually a measure of the viability of the cells²².

### Protocol

Mouse fibroblast cell line 3T3 was cultured in DMEM, supplemented with 5% of FBS, 100 IU/ml of penicillin and 100 μg/ml of streptomycin, and kept at 37° C. in 5% CO₂ incubator. For the preparation of cell culture 100 μL/well of cell solution (5×10⁴ cells/ml) was added into 96-well plate. The plate was incubated for overnight, and fresh medium was added after the removal of old medium. The test compounds were also added in different concentrations into the plate and plate was again incubated for 48 hrs. After the completion of this incubation period 200 μL MTT (0.5 mg/ml) was added and plate was again incubated for 4 hrs, after this final incubation 100 μL of DMSO was added to each well. The level of MTT reduction to formazan was evaluated by change in absorbance at 540 nm using a micro plate reader (Spectra Max plus, Molecular Devices, CA, USA). The cytotoxicity was recorded as concentration of the inhibitor causing 50% growth inhibition (IC₅₀) for 3T3 cells.

TABLE 2
Cytotoxicity of compound 1-19

| Compound | IC₅₀ ± SD [μM] |
|---|---|
| 1 | >30 |
| 2 | >30 |
| 3 | >30 |
| 4 | >30 |
| 5 | >30 |
| 6 | >30 |
| 7 | >30 |
| 8 | >30 |
| 9 | >30 |
| 10 | >30 |
| 11 | >30 |
| 12 | >30 |
| 13 | >30 |
| 14 | >30 |
| 15 | >30 |
| 16 | >30 |
| 17 | >30 |
| 18 | >30 |
| 19 | >30 |
| Standard | 0.26 ± 0.1 |
| (cyclohexamide) | |

### Mechanism of Action of Inhibitors

Kinetics studies were performed for each analogue. The assay was performed with different concentrations of compounds (0.2-0.00625 μM) over different concentration of chromogenic substrate 4-nitrophenyl acetate (0.175-1.40 mM). The compound 1 was un-substituted derivative and bind with the enzyme non-covalently through the allosteric site, noncompetitive inhibition is observed. Interestingly, compound 19 is substituted with Cl— groups and has shown mixed-type inhibition which is the indication that due to strong electron donating effect of Cl— groups on the benzamide aromatic system has enhanced the inhibition potential by interact with the enzyme through allosteric as well as active site. The Grafit version 7.0 was used for the construction of Lineveawer-Burk plots and their secondary replots. The different kinetics parameters for each analogue are shown in Table-3

TABLE 3
Kinetics Parameters of Benzamide analogue
against Bovine Carbonic Anhydrase-II.

| | | | | Vmax | | |
|---|---|---|---|---|---|---|
| | | Km | Vmax | app | | |
| | Km | app | (μM/L/ | (μM/L/ | Ki | Type of |
| No. | mM | mM | min)⁻¹ | min)⁻¹ | μM | Inhibition |
| 1 | 4.50 | 4.64 | 99.00 | 14.47 | 0.052 | Noncompetitive |
| 2 | 2.65 | 2.13 | 80 | 18.45 | 0.11 | Noncompetitive |
| 3 | 2.65 | 5.95 | 80 | 52.63 | 0.058 | Mixed type |
| 4 | 1.56 | 1.35 | 53.19 | 18.97 | 0.22 | Noncompetitive |
| 5 | 2.34 | 3.13 | 76.33 | 25.83 | 0.25 | Noncompetitive |
| 6 | 2.54 | 4.55 | 82.64 | 17.48 | 0.09 | Mixed type |
| 7 | 2.22 | 3.98 | 71.42 | 19.30 | 0.14 | Mixed type |
| 8 | 2.66 | 1.38 | 81.30 | 7.77 | 0.035 | Mixed type |
| 9 | 2.85 | 3.58 | 90.90 | 14.61 | 0.058 | Noncompetitive |
| 10 | 4.13 | 5.2 | 62.5 | 10.61 | 0.067 | Noncompetitive |
| 11 | 5.56 | 4.58 | 88.49 | 35.21 | 0.28 | Noncompetitive |
| 12 | 6.73 | 3.94 | 108.69 | 34.12 | 0.024 | Mixed type |
| 16 | 3.92 | 3.85 | 72.46 | 37.31 | 0.40 | Noncompetitive |
| 17 | 5.96 | 12.19 | 111.11 | 26.88 | 0.059 | Mixed type |
| 18 | 1.80 | 2.02 | 42.73 | 12.93 | 0.16 | Mixed type |
| 19 | 3.69 | 4.78 | 79.36 | 12.33 | 0.046 | Mixed type |
| ACZ | 3.5 | 3.5 | 48.89 | 14.22 | 0.05 | Noncompetitive |

### Calculations of Inhibitory Activities

The enzyme inhibitory activities were calculated using the following formula:

% Inhibition=100−(O.D of test/O.D of control)*100

Where test is the enzyme activity with sample and control is the enzyme activity without sample. The IC₅₀ of the compounds were calculated by using EZ-Fit Enzyme Kinetic Program (Perrella Scientific Inc. Amhrest, U.S.A).

1. A compound with inhibitory activity against carbonic anhydrase-II enzyme selected from the group consisting of:
N-(4-Sulfamoylphenylcarbamothioyl)benzamide;
4-Chloro-N-(4-sulfamoylphenylcarbamothioyl)benzamide;
4-Methyl-N-(4-sulfamoylphenylcarbamothioyl)benzamide;
4-Nitro-N-(4-sulfamoylphenylcarbamothioyl)benzamide;
3-Chloro-N-(4-sulfamoylphenylcarbamothioyl)benzamide;
3-Nitro-N-(4-sulfamoylphenylcarbamothioyl)benzamide;
2-Nitro-N-(4-sulfamoylphenylcarbamothioyl)benzamide;
2-Phenyl-N-(4-sulfamoylphenylcarbamothioyl)acetamide;
2-Methyl-N-(4-sulfamoylphenylcarbamothioyl)benzamide;
3-Methyl-N-(4-sulfamoylphenylcarbamothioyl)benzamide;
3-Methoxy-N-(4-sulfamoylphenylcarbamothioyl)benzamide;
4-Chloro-2-nitro-N-(4-sulfamoylphenylcarbamothioyl)benzamide;
4-Fluoro-N-(4-sulfamoylphenylcarbamothioyl)benzamide;
3,4,5-Trimethoxy-N-(4-sulfamoylphenylcarbamothioyl)benzamide;
2-Chloro-4-nitro-N-(4-sulfamoylphenylcarbamothioyl)benzamide;
3,5-Dimethoxy-N-(4-sulfamoylphenylcarbamothioyl)benzamide;
N-(4-Sulfamoylphenylcarbamothioyl)tetrahydrofuran-2-carboxamide;
2-Phenyl-N-(4-sulfamoylphenylcarbamothioyl)butanamide; and
2,4-Dichloro-N-(4(4-sulfamoylphenylcarbamothioyl)benzamide.

2. The compound of claim 1, wherein the compound is derived from a bovine source or manufactured by a recombinant process.

3. (canceled)
